(19) United States
(12) Patent Application Publication
(10) Pub. No.: US 2026/0043813 A1
(43) Pub. Date: Feb. 12, 2026

(54) Chronic kidney disease detection

(71) Applicant: The University Court of the University of Edinburgh, Edinburgh (GB)

(72) Inventors: David Baird, Edinburgh (GB); David Ferenbach, Edinburgh (GB)

(21) Appl. No.: 19/069,559

(22) Filed: Mar. 4, 2025

(30) Foreign Application Priority Data
Mar. 4, 2024 (GB) 2403125.4

(51) Int. Cl. G01N 33/68

(52) U.S. Cl. CPC G01N 33/6893; G01N 2333/78; G01N 2800/347; G01N 2800/50; G01N 2800/52; G01N 2800/56

(57) ABSTRACT

The disclosure provides biomarkers of cellular senescence for risk stratification and the identification of individuals at high risk of chronic kidney disease (CKD). The biomarkers may be used to identifying individuals who may or are likely to exhibit fast progression of chronic kidney disease, to facilitate the identification of individuals who are likely to respond to a targeted pharmacological intervention and/or to monitor outcomes of an intervention using a non-invasive approach.

### FIELD

The present disclosure relates to novel biomarkers which find utility in evaluating renal cellular senescence and/or kidney disease. Specifically, provided herein are uses, methods and kits comprising the novel biomarkers of the present disclosure.

### BACKGROUND

Chronic kidney disease (CKD) affects 850 million people worldwide (1) with kidney fibrosis the final common pathway regardless of the underlying aetiology. Senescent cells (SCs) are transcriptionally altered, permanently growth arrested cells which release multiple pro-inflammatory mediators that propagate injury; collectively termed the senescence associated secretory phenotype (SASP). Accumulating evidence has demonstrated senescence is an important driver of kidney fibrosis, with the epithelial cells of the renal tubule playing a central role (2). SC depletion in aged mice prolongs lifespan and improves the health of multiple organs. In the murine kidney, depletion of p21 positive SCs kidney using senolytic medication reduces fibrosis and improves renal regenerative capacity after injury (3). At present, there are no validated non-invasive biomarkers of senescence, with current techniques requiring tissue biopsies. This represents a key limitation for studies both underway and planned assessing senolytic drugs as treatments in human kidney disease. Previous studies have attempted to characterise the secretome of the senescent cells in vitro. The SASP atlas (4), utilised data derived from the proteomic analysis of supernatant from renal epithelial cells, comparing cells that had been irradiated to induce senescence to proliferating controls.

While various biomarkers have been implicated in chronic kidney disease, there is an unmet need to identify reliable non-invasive biomarkers that can be used as an indicator of renal senescence and/or kidney disease.

### SUMMARY

The present disclosure is based at least in part on the identification of novel biomarkers of cellular senescence which may find utility in aiding risk stratification and identifying individuals at high risk of chronic kidney disease (CKD). Specifically, the biomarkers detailed herein may find utility in identifying individuals who may or are likely to exhibit fast progression of chronic kidney disease. Further, the markers disclosed herein may be particularly useful in determining individuals who are likely to respond to a targeted pharmacological intervention and/or monitor outcomes of an intervention using a non-invasive approach. The present inventors sought to identify cellular senescence biomarkers indicative of renal disease which may be detected in readily available, non-invasive biological sources while being able to deliver rapid and reliable results.

In a first aspect, there is provided use of Clusterin and/or Fibronectin 1 as renal cellular senescence biomarker(s) for evaluating chronic kidney disease (CKD), wherein the level of Clusterin and/or Fibronectin 1 in a fluid sample from a subject is indicative of renal senescent cell load of said subject.

Throughout the present disclosure, the term “Fibronectin 1”, which is encoded by the FN1 gene, may be used interchangeably and/or is synonymous with the term “Fibronectin”.

In a second aspect of the present disclosure, there is provided a method of evaluating chronic kidney disease (CKD), said method comprising determining the level of one or more renal cellular senescence biomarkers in a fluid sample, wherein said one or more renal cellular senescence biomarkers comprise Clusterin and/or Fibronectin 1, and wherein the level of said one or more renal cellular senescence biomarkers is indicative of renal senescent cell load.

In a third aspect of the present disclosure, there is provided a kit for evaluating chronic kidney disease (CKD), said kit comprising one or more probes for the detection of one or more renal cellular senescence biomarkers, wherein said one or more renal cellular senescence biomarkers comprise Clusterin and/or Fibronectin 1.

Throughout the present disclosure references are made to a number of terms which are to be understood to have the meanings provided below, unless a context indicates to the contrary.

The terms “comprise”, “comprising” and/or “comprises” are used to denote aspects and embodiments of this invention that “comprise” a particular feature or features. It should be understood that these terms may also encompass aspects and/or embodiments which “consist essentially of” or “consist of” the relevant feature or features.

The term “chronic kidney disease” typically refers to a chronic condition characterised by a persistent loss of renal function, which can progress to end-stage kidney disease. End-stage kidney disease requires renal replacement therapy in the form of dialysis or a kidney transplant. Without wishing to be bound by theory, chronic kidney disease is thought to occur as a maladaptive response to diverse forms of acute and chronic injury, wherein kidney fibrosis is implicated regardless of the underlying aetiology.

Again, without wishing to be bound by theory, estimated glomerular filtration rate (eGFR) is typically used to define and stage chronic kidney disease in the art. Chronic kidney disease may be broadly divided into five stages. Stages 1 to 2 typically refer to mild kidney damage (stage 1: eGFR of 90 or higher; stage 2: eGFR: 60-89), stage 3a typically refers to mild to moderate kidney damage (eGFR: 45-59), stage 3b typically refers to moderate to severe damage (eGFR: 30-44), stage 4 typically refers to severe kidney damage (eGFR: 15-29) and stage 5 typically refers to most severe kidney damage (eGFR<15).

The term “evaluating chronic kidney disease” as used herein may encompass evaluating or determining any one or more of the following:
(i) decline in renal function;
(ii) the likelihood of developing chronic kidney disease;
(iii) the severity of chronic kidney disease;
(iv) progression of chronic kidney disease;
(v) the likelihood of progression of chronic kidney disease; and/or
(vi) the efficacy or effectiveness of a treatment, therapy or drug for a kidney disease.

In some examples, “evaluating chronic kidney disease” may refer to evaluating, determining or identifying subjects who are likely to exhibit fast progression of chronic kidney disease. In some examples, “evaluating chronic kidney disease” may refer to evaluating the likelihood the subject will progress to the next stage of kidney disease, wherein each stage may be defined by eGFR values as detailed above.

Accordingly, in some examples, “evaluating chronic kidney disease” may refer to evaluating the likelihood the subject will progress from stage 1 to any of stages 2-5 of kidney disease as defined above. In some examples, “evaluating chronic kidney disease” may refer to evaluating the likelihood the subject will exhibit fast progression from stage 1 to any of stages 2-5 of kidney disease. In one example, “evaluating chronic kidney disease” may refer to evaluating the likelihood the subject will progress or will exhibit fast progression from stage 1 to stage 2 of kidney disease.

In a further example, “evaluating chronic kidney disease” may refer to evaluating the likelihood the subject will progress from stage 2 to any of stages 3a-5 of kidney disease. In another example, “evaluating chronic kidney disease” may refer to evaluating the likelihood the subject will exhibit fast progression from stage 2 to any of stages 3a-5 of kidney disease. In one example, “evaluating chronic kidney disease” may refer to evaluating the likelihood the subject will progress or will exhibit fast progression from stage 2 to stage 3a of kidney disease.

In a further example, “evaluating chronic kidney disease” may refer to evaluating the likelihood the subject will progress from stage 3a to any of stages 3b-5 of kidney disease. In another example, “evaluating chronic kidney disease” may refer to evaluating the likelihood the subject will exhibit fast progression from stage 3a to any of stages 3b-5 of kidney disease. In one example, “evaluating chronic kidney disease” may refer to evaluating the likelihood the subject will progress or will exhibit fast progression from stage 3b to stage 4 of kidney disease.

In yet another example, “evaluating chronic kidney disease” may refer to evaluating the likelihood the subject will progress from stage 3b to stages 4 or 5 of kidney disease. In another example, “evaluating chronic kidney disease” may refer to evaluating the likelihood the subject will exhibit fast progression from stage 3b to stages 4 or 5 of kidney disease. In one example, “evaluating chronic kidney disease” may refer to evaluating the likelihood the subject will progress or will exhibit fast progression from stage 4b to stage 5 of kidney disease.

In yet another example, “evaluating chronic kidney disease” may refer to evaluating the likelihood the subject will progress from stage 4 to stage 5 of kidney disease. In another example, “evaluating chronic kidney disease” may refer to evaluating the likelihood the subject will exhibit fast progression from stage 4 to stage 5 of kidney disease.

Further, “evaluating or determining” chronic kidney disease may comprise, consist essentially of or consist of detecting the presence or determining the level of any one or more biomarkers disclosed herein. In a preferred example, evaluating chronic kidney disease may comprise, consist essentially of or consist of determining the level of any one or more biomarkers disclosed herein.

Accordingly, “evaluating chronic kidney disease” may involve evaluating any one or more of:
(i) decline in renal function;
(ii) the likelihood of developing chronic kidney disease;
(iii) the severity of chronic kidney disease;
(iv) progression of chronic kidney disease;
(v) the likelihood of progression of chronic kidney disease; and/or
(vi) the efficacy or effectiveness of a treatment, therapy or drug for a kidney disease;
   by detecting the presence or determining the level of any one or more of the biomarkers disclosed herein.

In some examples, detecting the presence or determining the level of any one or more of the biomarkers disclosed herein may be assessed over time, such as over 1, 2, 3, 4, 5, 6, 7, 8, 9, 10, 15, 20, 25 or 30 year(s).

As it will be appreciated by the skilled person in the art, the term “biomarker” typically refers to an indicator of a specific biological state which may act as surrogates for risk, presence or stage of a disease. Biomarkers may be particularly useful when detecting a condition, monitoring disease progression and/or monitoring response to therapeutic interventions. Thus, biomarkers typically need to be sensitive for a particular condition such that the condition may be detected early, and be able to delineate subjects with the conditions and healthy subjects. In the present context, biomarkers may embrace indicators of renal function and/or kidney disease, such as any of the examples (i) to (vi) provided above.

In relation to any of the aspects of the present disclosure, evaluating chronic kidney disease may comprise, consist essentially of or consist of determining the level of Clusterin and/or Fibronectin 1 in a sample, and comparing the level(s) to a threshold range or value(s). In a preferred teaching of the present disclosure, the level of any one or more of the biomarkers disclosed herein may correlate with the level of senescent cells in the kidneys and/or may be an indicator of aberrant accumulation of senescent cells in the kidneys (i.e. “indicative of renal senescent cell load”). Without wishing to be bound by theory, senescent cells accumulate in a number of renal diseases and are associated with negative kidney function and outcome.

In one example, determining the level of any one or more biomarkers disclosed herein may comprise, consist essentially of or consist of measuring the concentration of said one or more biomarkers in a sample. In some examples, determining the level of any one more biomarkers disclosed herein may comprise, consist essentially of or consist of comparing the amount or concentration of said one or more biomarkers in a sample to a reference marker or value(s). In a preferred example, determining the level of any one more biomarkers disclosed herein, such as Clusterin and/or Fibronectin 1, may comprise, consist essentially of or consist of normalising the amount of said one or more biomarkers against an amount of another biomarker in said sample, such as an alternative marker of urinary concentration.

By way of example, the term normalising as used herein typically refers to obtaining a ratio between the amount or concentration of a biomarker of interest and the amount or concentration of a marker of urinary concentration (e.g. Creatinine and/or Cystatin C).

In a specific example, determining the level of any one or more biomarkers disclosed herein may comprise, consist essentially of or consist of normalising the amount or concentration of Clusterin and/or Fibronectin 1 in a sample against the amount or concentration of Creatinine and/or Cystatin C. In a further example, determining the level of any one or more biomarkers disclosed herein may comprise, consist essentially of or consist of normalising the amount or concentration of Clusterin and/or Fibronectin 1 in a sample against the amount or concentration of Creatinine. In a further example, determining the level of any one or more biomarkers disclosed herein may comprise, consist essentially of or consist of normalising the amount or concentration of Clusterin and/or Fibronectin 1 in a sample against the amount or concentration of Cystatin C.

As detailed above, the level of Clusterin and/or Fibronectin 1 may be normalised or compared with a standard value or a reference value. The standard value or reference value may be an amount or concentration of another marker, such as Creatinine and/or Cystatin C. in some examples, the standard value or reference value may be obtained from the same sample of interest.

In addition, or alternatively, the standard value or the reference value may be obtained from a reference sample or cohort.

In one teaching, the level of Clusterin and/or Fibronectin 1 may be compared with a threshold range or value(s). A threshold value may be calculated as part of a step of determining the level of one or more renal cellular senescence biomarkers. A threshold value may, as stated, comprise the determination of a ratio of the amount or concentration of a biomarker of interest and the amount or concentration of a marker of urinary concentration (e.g. Creatinine and/or Cystatin C). In one teaching, the ratio may comprise the ratio of Clusterin (and/or Fibronectin):Creatinine (and/or Cystatin C). The ratio may be expressed as an amount (for example a μg amount) of Clusterin or fibronectin per amount (for example mmol amount) of Creatinine or Cystatin C. By way of example, ratios of Clusterin (and/or fibronectin) to Creatinine (and/or Cystatin C) may comprise any ratio that indicates, is diagnostic of or is associated with a chronic kidney disease (CKD). A suitable ratio may span any range suitable for assessing senescent cell load and the risk of fast progression of CKD as disclosed herein. It should be noted that the precise value or range of suitable ratios may vary depending on, for example the subject, the sample and/or the stage or progression of any associated chronic kidney disease. Moreover, the skilled person will be aware of other ways in which an amount or level of Clusterin and/or Fibronectin 1 may be determined to serve as renal cellular senescence biomarker(s) for evaluating chronic kidney disease (CKD).

In relation to any of the aspects provided herein, example threshold ranges of Clusterin as disclosed herein may comprise, consist essentially of or consist of any one of the ranges provided below (μg Clusterin per mmol Creatinine):
(a) 100-200 μg/mmol;
(b) 110-170 μg/mmol;
(c) 120-160 μg/mmol;
(d) 110-130 μg/mmol; or
(e) 140-160 μg/mmol.

As such a sample found to contain an amount of Clusterin:Creatinine (e.g. μg Clusterin per mmol Creatinine) according to the ranges set out in (a)-(e) above, may be a sample provided by or obtained from, a subject (for example a human or animal subject): suffering from (or predisposed/susceptible to or at (high) risk of developing) a chronic kidney disease; likely to exhibit fast progression of chronic kidney disease and/or likely to respond to a targeted pharmacological intervention. Further diagnostic ranges (for example, ranges of Clustering to Creatinine) suitable for evaluating chronic kidney disease (CKD) are set out below.

In one example, the threshold range of Clusterin as disclosed herein may comprise or consists of 10-1000 μg/mmol (μg Clusterin per mmol Creatinine).

In one example, the threshold range of Clusterin as disclosed herein may comprise or consists of 40-200 μg/mmol (μg Clusterin per mmol Creatinine).

In a preferred example, the threshold range of Clusterin as disclosed herein may comprise, consist essentially of or consist of 120-160 μg/mmol (μg Clusterin per mmol Creatinine).

In one example, the threshold value of Clusterin as disclosed herein may comprise, consist essentially of or consist of 110, 120, 121, 122, 123, 124, 125, 126, 127, 128, 129, 130 130, 135, 140, 145, 150, 151, 152, 153, 154, 155, 156, 157, 158, 159, 160 or 170 μg/mmol (μg Clusterin per mmol Creatinine).

In some examples, the threshold value of Clusterin as disclosed herein may comprise or consist of 50, 51, 52, 53, 53, 55, 56, 57, 58, 59, 60, 61, 62, 63, 64, 65, 66, 67, 68, 69, 70, 100, 101, 102, 103, 104, 105, 106, 107, 108, 109, 110, 111, 112, 112.5, 113, 114, 115, 116, 116, 118, 119, 120, 121, 122, 123, 124, 124.5, 125, 126, 127, 128, 129 or 130 μg/mmol (μg Clusterin per mmol Creatinine).

In relation to any of the aspects provided herein, the threshold range or value(s) of Fibronectin 1 as disclosed herein may comprise, consist essentially of or consist of any one of the ranges provided below (μg Fibronectin 1 per mmol Creatinine):
(a) 10-100 μg/mmol;
(b) 20-60 μg/mmol;
(c) 30-50 μg/mmol;
(d) 20-40 μg/mmol; or
(e) 40-60 μg/mmol.

As such a sample found to contain an amount of Fibronectin:Creatinine (e.g. μg Fibronectin per mmol Creatinine) according to the ranges set out in (a)-(e) above, may be a sample provided by or obtained from a subject (for example a human or animal subject): suffering from (or predisposed/susceptible to or at (high) risk of developing) a chronic kidney disease; likely to exhibit fast progression of chronic kidney disease and/or likely to respond to a targeted pharmacological intervention. Further diagnostic ranges (for example, ranges of fibronectin to Creatinine) suitable for evaluating chronic kidney disease (CKD) are set out below.

In one teaching, the threshold range of Fibronectin as disclosed herein may comprise, consist essentially of or consist of 15 to 260 μg/mmol (μg Fibronectin 1 per mmol Creatinine).

In a preferred example, the threshold range of Fibronectin 1 as disclosed herein may comprise, consist essentially of or consist of 30-50 μg/mmol (μg Fibronectin 1 per mmol Creatinine).

In one example, the threshold value of Fibronectin 1 as disclosed herein may comprise, consist essentially of or consist of 20, 25, 30, 31, 32, 33, 34, 35, 36, 37, 38, 39, 40, 41, 42, 43, 44, 45, 46, 47, 48, 49, 50, 55, 60, 65, 66, 67, 68, 69, 70 or 71 μg/mmol (μg Fibronectin 1 per mmol Creatinine).

As it will be appreciated by the skilled person in the art, the term “cellular senescence” as used herein typically refers to the state of irreversible cell cycle arrest, wherein the cell undergoes marked changes in transcriptional and secretory activity as well as modifications in morphology and chromatin organisation. Senescence is implicated in multiple contexts in the art, such as during development as well as in response to a range of insults (e.g. cellular stress, hypoxia, lack of nutrients, genotoxic injury, oncogene activation and mitochondrial dysfunction). Without wishing to be bound by theory, cellular senescence is thought to be an important driver of kidney fibrosis, wherein the epithelial cells of the renal tubule playing a central role.

The term “renal senescent cell load” may also be referred to as senescent cell burden, which typically refers to the accumulation of senescent cells in the kidneys. Renal senescent cell load typically refers to increased number of senescent cells in the kidneys, such as in the epithelium of renal tubules. Without wishing to be bound by theory, increased renal senescent cell load occurs in injury, disease and aging. Aberrant accumulation of senescent cells is implicated in impaired renal function and kidney disease.

In one example, renal senescent cell load may comprise, consist essentially of or consist of renal tubular epithelial senescent cell load.

The skilled person in the art would recognise various methods of detecting cellular senescence and/or cellular senescent cell load, such as in a tissue or an organ.

By way of example only, a first step of determining or evaluating renal senescent cell load may involve obtaining a tissue biopsy from an organ of interest (e.g. kidney), and detecting and/or assessing the expression of senescence-associated β-galactosidase activity (SA-β-GAL). Without wishing to be bound by theory, SA-β-GAL reflect the enhanced lysosomal content of senescent cells. However, SA-β-GAL cannot be used as a marker when the sample of interest is paraffin-embedded tissue sections or live cells. In addition, or alternatively, lipofuscin may be quantified by histochemical staining of tissues or cells.

As a second step of determining or evaluating renal senescent cell load, additional markers of senescence may be detected. Specifically, increased levels of expression of markers such as cyclin-dependent kinase inhibitors P16INK4A (encoded by the gene CDKN2A) and P21CIP1 (encoded by the gene CDKN1A) are associated with senescence. Without wishing to be bound by theory, P16INK4A and P21CIP1 are implicated in cell cycle arrest at the G1/S checkpoint and are reported to be increased in senescent cells. Further, senescent cells typically lack expression of or have reduced levels of cell proliferation and/or differentiation markers, such as Ki-67 and lamin-B1.

However, determining or evaluating cellular senescence using the markers known in the art described above typically require obtaining a tissue sample from the organ or tissue of interest and staining for various markers indicative of cellular senescence. This approach requires an invasive intervention of a subject (e.g. a biopsy) and thus is impractical when monitoring renal decline or chronic kidney disease in a subject over time or monitoring the efficacy of a therapeutic intervention in a subject over time. Moreover, the detection of any one of the reported markers alone may be insufficient to confirm senescence and false positives may occur. In view of this, there is a need to identify non-invasive biomarkers with high specificity and sensitivity.

As detailed herein, the present inventors have unexpectedly identified urinary Clusterin and/or Fibronectin 1 to be useful biomarkers which provides a non-invasive approach of evaluating decline in renal function and/or chronic kidney disease.

Without wishing to be bound by theory, Clusterin (encoded by the CLU gene) is a glycoprotein implicated as an extracellular chaperone protein involved in numerous cellular processes and has a purported anti-apoptotic role.

The present disclosure also provides Fibronectin 1 (encoded by the FN1 gene) as a useful marker of renal cellular senescence. Without wishing to be bound by theory, Fibronectin 1 is involved in various cellular roles, such as adhesion, growth, migration and differentiation, and is reported to be secreted by multiple cell types, including fibroblasts.

Again, without wishing to be bound by theory, it is envisaged that these biomarkers alone, or in combination with other biomarkers known in the art, find utility as predictors or indicators of renal disease.

In the first or second aspect of the present disclosure, the use or method as detailed herein may comprise, consist essentially of or consist of up to 10 additional biomarkers. In some examples, the use or the method as detailed herein may comprise, consist essentially of or consist of up to 5 additional biomarkers. In one teaching, the use or the method of the present disclosure may comprise, consist essentially of or consist of 1, 2, 3, 4, 5, 6, 7, 8, 9 or 10 additional biomarker(s). For the avoidance of doubt, the use or method comprising or consisting of additional biomarker(s) means the use or method must include Clusterin and/or Fibronectin 1.

In some examples, the use consists of or consists essentially of Clusterin and/or Fibronectin 1.

In some examples, the method of evaluating chronic kidney disease, said method comprising, consisting essentially of or consisting of determining the level of one or more renal cellular senescence biomarkers in a fluid sample, wherein said one or more renal cellular senescence biomarkers consist essentially of or consist of Clusterin and/or Fibronectin 1, and wherein the level of said one or more renal cellular senescence biomarkers is indicative of renal senescent cell load.

As stated, the present inventors sought to identify biomarkers that may be detected in readily available, non-invasive biological samples while being able to deliver rapid and reliable results. As such, the present disclosure preferably involves detecting the level of any one or more of the biomarkers disclosed herein in a fluid sample obtained from a subject.

The term “fluid sample” as used herein embraces any fluid sample obtained from a subject, such as blood, plasma, serum or urine, for example. The use of a fluid sample is particularly advantageous as it enables a sample to be obtained from an individual in a non-invasive manner.

As stated, in one teaching, the fluid sample may comprise, consist essentially of or consist of urine, blood, serum or plasma. In a preferred embodiment, the fluid sample comprises or consists of a urine sample.

The term “subject” typically refers to any subject predisposed, susceptible or at risk of developing or exhibiting decline in renal function, kidney injury, kidney disease, acute kidney injury and/or chronic kidney disease. In some examples, the subject of the present disclosure may not exhibit signs of overt kidney disease—for example, the subject may not yet exhibit known signs or symptoms of decline in kidney function and/or kidney disease. In some examples, the subject of the present disclosure is a subject with stage 1 kidney disease or with at least stage 1 of kidney disease. In other examples, the subject is a subject with stage 2, 3a or 3b kidney disease. In other examples, the subject is a subject with at least stage 2, 3a or 3b kidney disease. The subject may also encompass any subject who has developed decline in renal function, kidney injury, kidney disease, acute kidney injury and/or chronic kidney disease. In some examples, the subject may be undergoing treatment or a therapeutic/pharmacological intervention for the prevention and/or treatment of a kidney disease. In some examples, the subject may be undergoing treatment or a therapeutic/pharmacological intervention to delay progression of kidney disease.

For the avoidance of doubt, the term “subject” may encompass any animal or human subject. In a preferred teaching, the subject embraces any mammalian subject. In yet another preferred example, the subject embraces a human subject. In some examples the subject may be a dog or a cat.

As stated, in relation to the third aspect of the present disclosure, there is provided a kit for evaluating chronic kidney disease.

The kit of the present disclosure comprises one or more probes for the detection of one or more renal cellular senescence biomarkers, wherein said one or more biomarkers comprise, consist essentially of or consist of Clusterin and/or Fibronectin 1.

In one teaching, the kit comprises or consists of one or more probes for the detection of Clusterin and/or Fibronectin 1, and up to 10 additional biomarkers. In yet another example, the kit comprises or consists of one or more probes for the detection of Clusterin and/or Fibronectin 1, and up to 5 additional biomarkers.

In some examples, the kit comprises or consists of one or more probes for the detection of Clusterin and/or Fibronectin 1, and 1, 2, 3, 4, 5, 6, 7, 8, 9 or 10 additional biomarker(s).

In one example, there may be one probe for the detection of each renal cellular senescence biomarker and/or each additional biomarker. In one example, there may be more than one probe for the detection of each renal cellular senescence biomarker and/or each additional biomarker.

As described above, the additional biomarkers may comprise, consist essentially of or consist of any one or more of the following:
(i) Cystatin C;
(ii) Kidney injury molecule-1 (KIM1);
(iii) Epidermal growth factor (EGF);
(iv) Monocyte chemoattractant protein 1;
(v) Transforming growth factor alpha;
(vi) Neutrophil Gelatinase-associated Lipocalin (NGAL);
(vii) Growth and Differentiation Factor 15 (GDF15);
(viii) Fatty Acid Binding Protein (FABP);
(ix) Osteopontin;
(x) Tissue Inhibitor of Metalloprotease 1 (TIMP1);
(xi) Uromodulin;
(xii) Vascular Endothelial Growth Factor A (VEGFA);
(xiii) Interleukin-6 (IL6);
(xiv) Leukaemia Inhibitory Factor (LIF);
(xv) Matrix Metallopeptidase 9 (MMP9);
(xvi) Albumin;
(xvii) Creatinine.

In one example, the additional biomarkers may comprise or consist of:
(i) Cystatin C;
(ii) Kidney injury molecule-1 (KIM1);
(iii) Epidermal growth factor (EGF);
(iv) Monocyte chemoattractant protein 1;
(v) Transforming growth factor alpha;
(vi) Neutrophil Gelatinase-associated Lipocalin (NGAL).

In some examples, the kit for evaluating chronic kidney disease of the present disclosure comprises one or more probes for the detection of one or more renal cellular senescence biomarkers, wherein said one or more renal cellular senescence biomarkers consist of or consist essentially of Clusterin and/or Fibronectin 1.

A suitable probe for the detection of any of the biomarkers described herein may embrace any probe specifically designed to detect the presence and/or level of one or more biomarkers of interest, such as an antibody, a conjugated antibody or any suitable fluorescent probes known in the art.

In certain examples, the kit may comprise an immunoassay kit. For example, the kit may comprise one or more primary antibodies which specifically bind an epitope of a biomarker of interest. The primary antibodies may optionally be conjugated to a detectable moiety, such as a fluorescent probe. Alternatively, the kit may be suitable for a sandwich method wherein the primary antibody binds to a biomarker present in a fluid sample, and a secondary antibody conjugated to a detectable moiety binds said primary antibody. In order to generate a detectable signal, antibodies may be conjugated to a fluorescent moiety, electrochemical label, electrochemical luminescence label, metal chelate, colloidal metal particle, biotin, streptavidin, enzymes (e.g. horseradish peroxidase, alkaline phosphatase), for example. Numerous immunoassay formats are known in the art which may be compatible with fluid samples.

In some examples, the kit may comprise an immunoassay plate (such as an ELISA plate) or a lateral flow-type device. For example, the fluid sample may be contacted with an immunoassay plate or a lateral flow device whereupon one or more detectable signal(s) is used to confirm the presence or absence of any of the biomarkers disclosed herein. In other examples, the kit may be suitable for a microfluidics-based multiplex immunoassay.

The kit disclosed herein may optionally further comprise a receptacle for a fluid sample. In one example, the receptacle is for a urine sample and/or a blood sample. In one example, the receptacle is for a urine sample.

In some examples, the kit may comprise a receptacle and one or more buffers or solutions. Such kits may be useful for delivering the fluid sample to a laboratory for testing, such as using one or more quantitative methods known in the art. By way of example only, a suitable quantitative method for the detection of one or more biomarkers of interest may include LC-MS and/or ELISA, for example.

In addition to the above, the kit of the present disclosure may further comprise one or more positive and/or negative controls.

A suitable positive control may comprise, consist essentially of or consist of any soluble sample containing a known quantity of a protein a biomarker. In one example, a positive control may comprise, consist essentially of or consist of a known quantity of a protein or a biomarker of interest in a reagent diluent (e.g. 1% BSA in PBS).

A suitable negative control may comprise, consist essentially of or consist of any solution which does not contain a biomarker of interest. In some examples, the negative control may be a blank solution.

Accordingly, in one example, a kit of the present disclosure may comprise, consist essentially of or consist of any one or more of the following components:
(i) a lateral flow device or a plate for an immunoassay, wherein said device or plate comprises one or more antibodies for the detection of Clusterin and/or Fibronectin 1; optionally, wherein said device or plate further comprises one or more antibodies for the detection of any one or more of biomarkers disclosed herein;
(ii) a negative control as disclosed herein;
(iii) a positive control as disclosed herein;
(iv) a receptacle;
(v) one or more solution(s) and/or buffer(s).

In one example, the kit provided above may comprise, consist essentially of or consist of 2 or more of the components (i) to (v). In some examples, the kit provided above may comprise, consist essentially of or consist of 3 or more of the components (i) to (v). In a further example, the kit provided above may comprise, consist essentially of or consist of 4 or more of the components (i) to (v). In a further example, the kit provided above may comprise, consist essentially of or consist of the components (i) to (v).

### DETAILED DESCRIPTION

The present disclosure will now be further described by way of example and with reference to the Figures, which show:

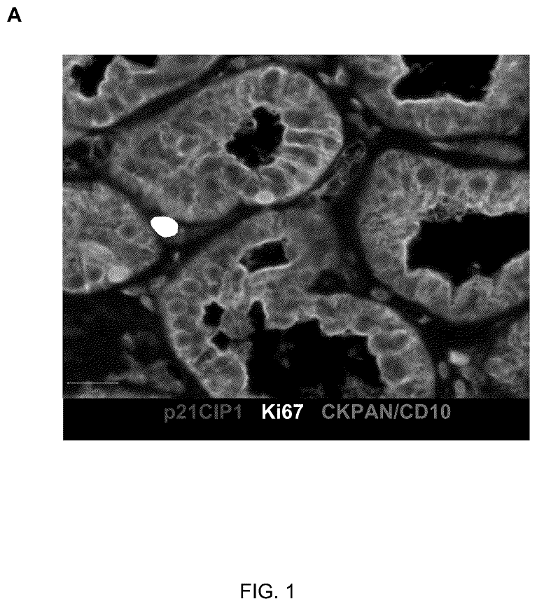

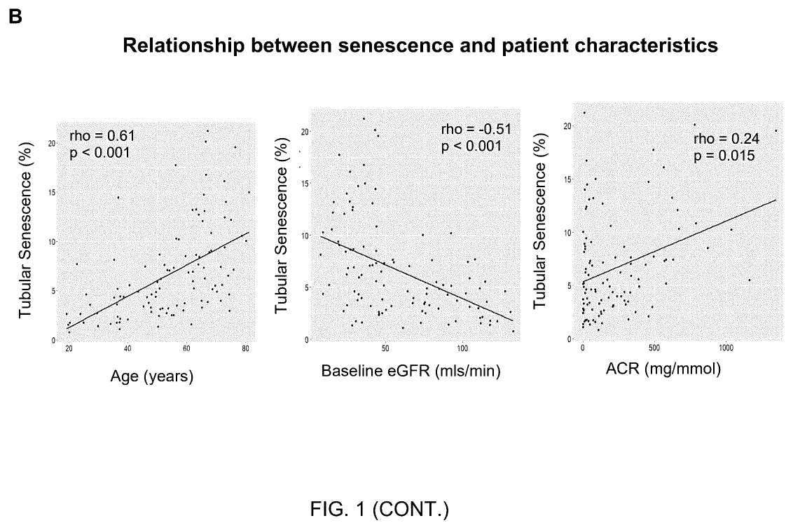

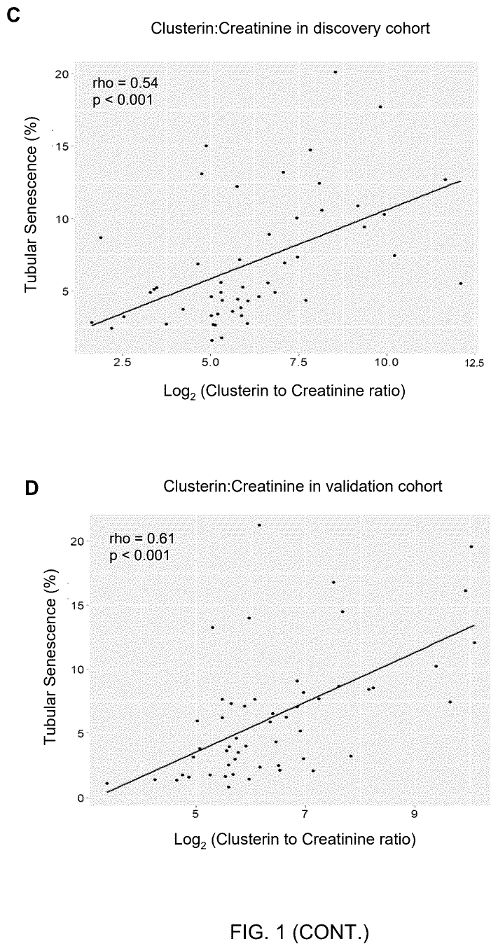

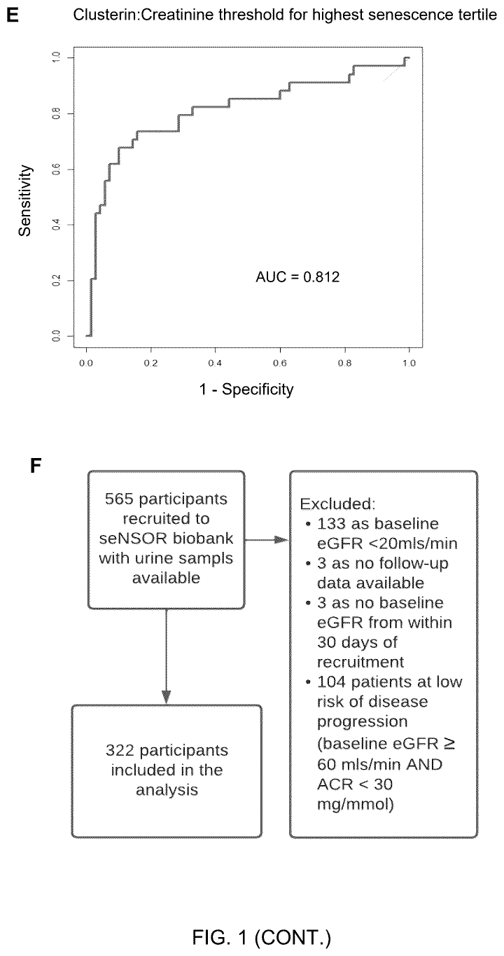

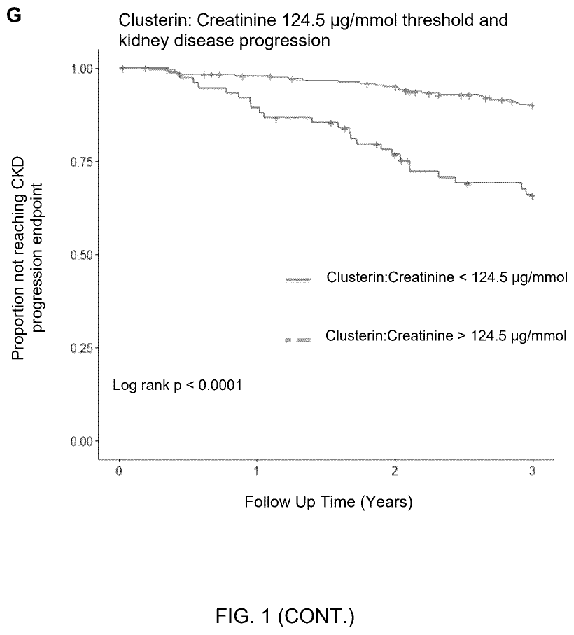

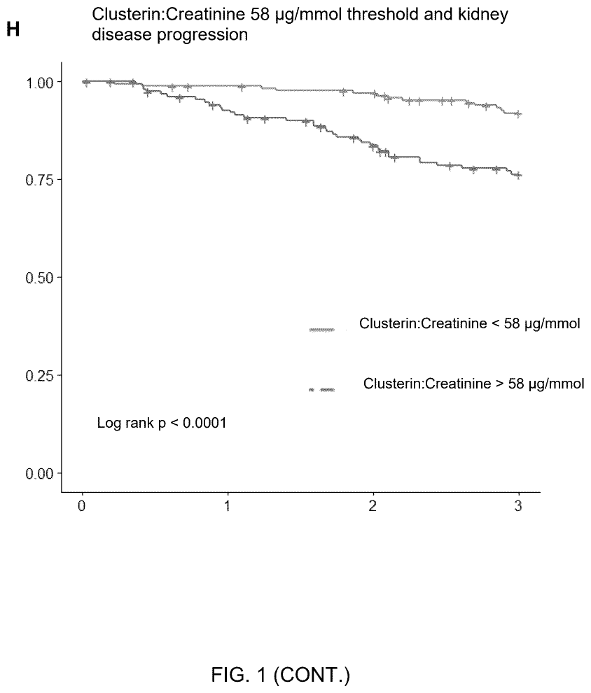

FIG. 1. Urinary Clusterin as a biomarker of human kidney senescence and predictor of CKD progression. (A) Representative image of immunofluorescent stain used quantify tubular senescence. (B) Relationship between histological senescence and age, renal function, and albuminuria (n=104, spearman's rank correlation). Correlation between histological senescence and urinary Clusterin:creatinine ratio in the discovery (C) and validation (D) cohorts (n=51 and n=53 respectively, spearman rank correlations). (E) Receiver operating curve using urinary Clusterin:creatinine ratio levels to discriminate between those with the highest tertile of histological senescence and the remaining participants (n=104). (F) Flowchart showing participants from seNSOR included in outcome analysis. (G) Kaplan Meier curves for progression of kidney disease high vs low urinary Clusterin:Creatinine ratio (cut off 124.5 μg/mmol, n=322). (H) Kaplan Meier curves for progression of kidney disease high vs low urinary Clusterin:Creatinine ratio (cut off 58 μg/mmol, n=322).

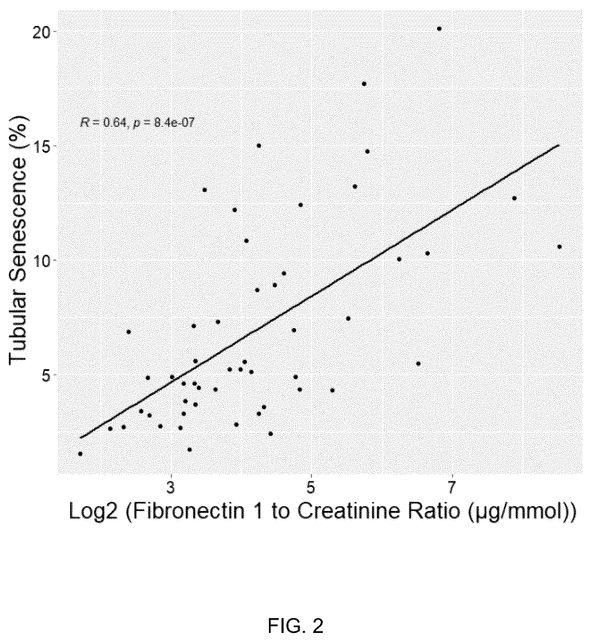

FIG. 2. Fibronectin 1 in the discovery cohort samples using an ELISA. Urinary Fibronectin 1:creatinine ratio correlates significantly with histological senescence (rho=0.64, p<0.001, n=51).

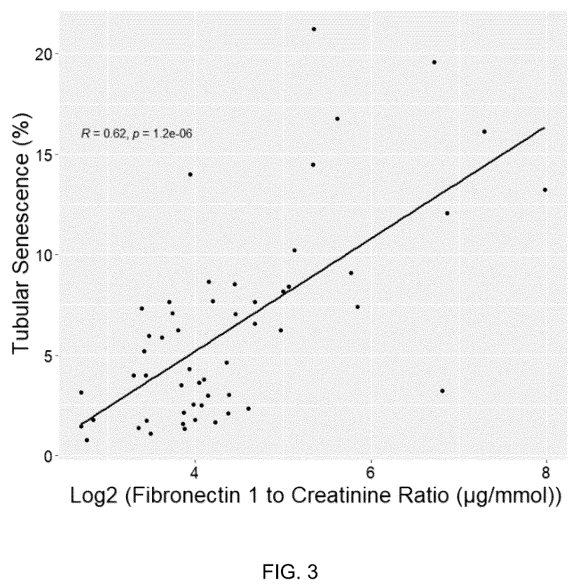

FIG. 3. Fibronectin 1 quantified in the validation cohort (n=53) using ELISA. Urinary Fibronectin 1:Creatinine ratio continues to correlate strongly with tubular senescence (rho=0.62, p<0.001, n=53).

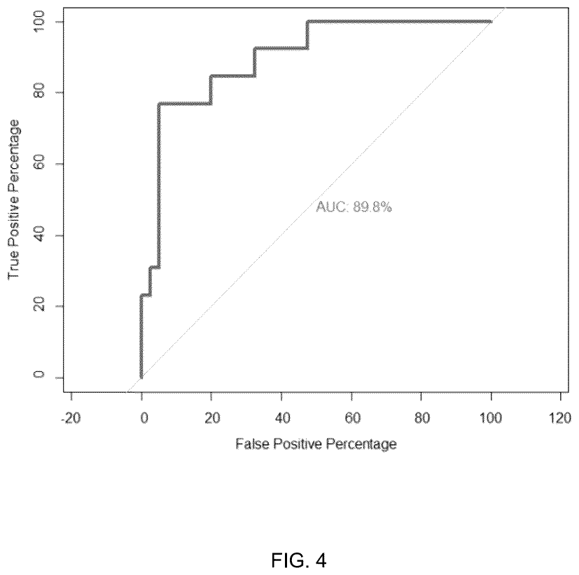

FIG. 4. Receiver operating curve using urinary Fibronectin1:creatinine levels to discriminate between those with the highest quartile of histological senescence and the remaining participants in the validation cohort (n=53).

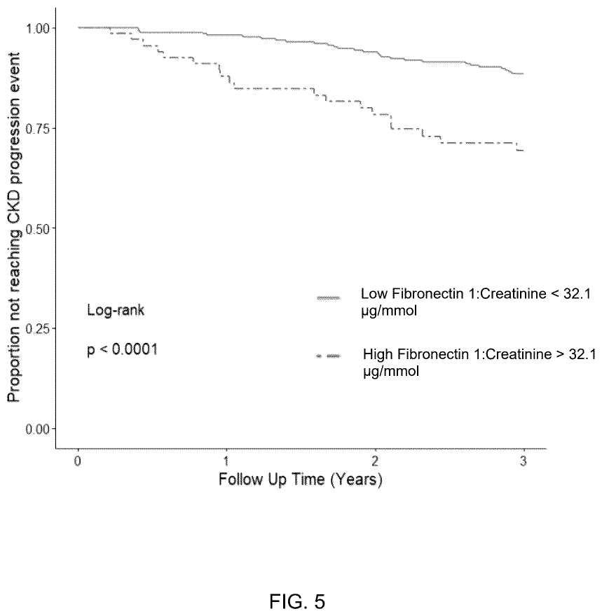

FIG. 5. Kaplan Meier curves for progression of kidney disease high vs low urinary Fibronectin 1:Creatinine (cut off 32.1 μg/mmol, n=322).

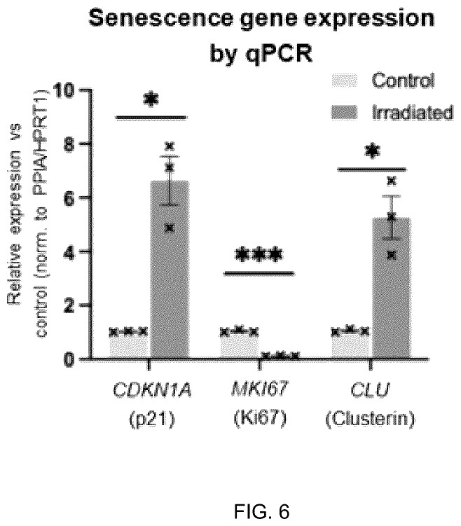

FIG. 6. Senescence gene expression by qPCR.

Relative expression of CDKN1A, MKI67 and CLU in irradiated hRPTECs (n=3) compared to proliferating controls (n=3). Normalised for HPRT1 and PPIA (average) expression. Individual data points plotted with mean and SEM. Welch's t-test performed.

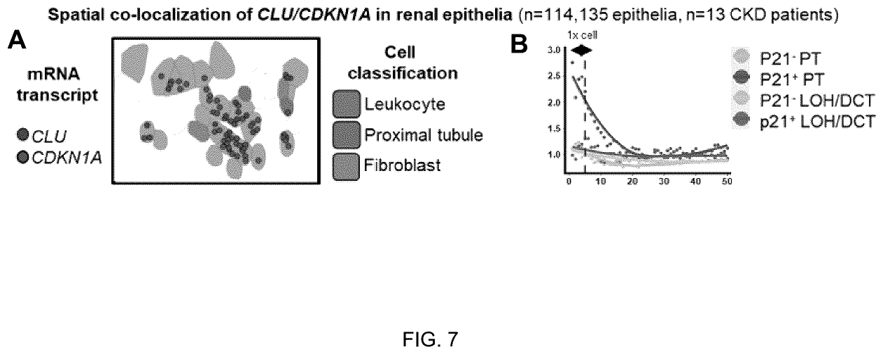

FIG. 7. Spatial co-localization of CLU/CDKN1A in renal epithelia.

(A) Image of CLU/CDKN1A co-localisation by spatial transcriptomic analysis. (B) Spatial enrichment (log 2 scale) of CLU transcripts in proximity to P21+ and P21-renal proximal tubule (PT) or loop of Henle/distal convoluted tubule (LOH/DCT) epithelial cells.

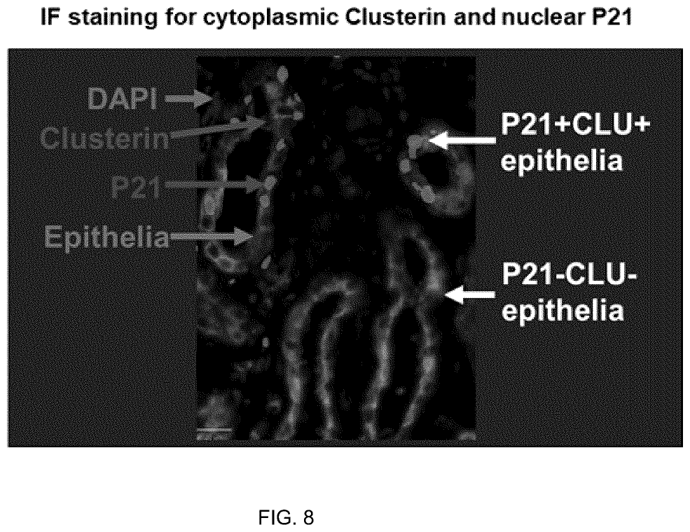

FIG. 8. Immunofluorescence staining for cytoplasmic Clusterin and nuclear P21. Immunofluorescent staining for P21 and Clusterin in human kidney biopsies with channels visible as per image label.

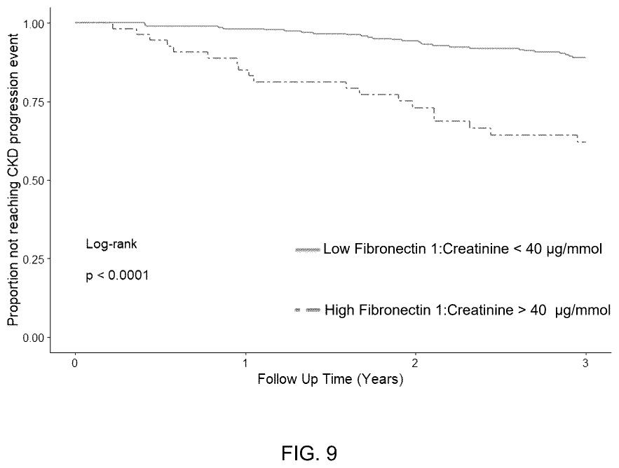

FIG. 9. Kaplan Meier curves for progression of kidney disease high vs low urinary Fibronectin 1:Creatinine (cut off 40 μg/mmol, n=322).

### EXAMPLE 1—URINARY CLUSTERIN PREDICTS LEVELS OF RENAL SENESCENCE

Human kidney and urine samples were obtained from patients with CKD from the seNSOR biobank to identify candidate urinary biomarkers of senescence. The most promising candidate, Clusterin, was then investigated further to determine its ability to predict renal progression in patients with kidney disease.

104 human kidney biopsies were obtained all with paired urine samples; 51 patients, recruited in Edinburgh, were our discovery cohort with the remaining 53 patients, recruited in Glasgow, our validation cohort. Baseline characteristics shown in Table 1.

TABLE 1
Baseline characteristics of the discovery and validation cohorts

| | | | p value for |
|---|---|---|---|
| | Discovery | Validation | comparison |
| N | 51 | 53 | |
| Age (years), median | 56.2 (45.6, 67.4) | 54.9 (37.1, 65.3) | 0.61 |
| (IQR) | | | |
| Male/Female, n | 32/19 | 29/24 | 0.53 |
| eGFR (ml/min/1.73 m²), | 45.6 (28.8, 83.8) | 47.1 (32.6, 84.2) | 0.61 |
| median (IQR) | | | |
| SBP, mean ± S.D | 139 ± 21.8 | 139.5 ± 20.5 | 0.9 |
| DBP, mean ± S.D | 80.2 ± 12.4 | 82.5 ± 13.6 | 0.37 |
| uACR (mg/mmol), | 85.9 (16.4, 247.7) | 131.2 (29.2, 333.2) | 0.14 |
| median (IQR) | | | |

Ethnicity

| | | |
|---|---|---|
| White, n | 47 | 43 |
| Black, n | 1 | 0 |
| Asian, n | 1 | 3 |
| Not recorded, n | 2 | 7 |

Diagnoses

| | | |
|---|---|---|
| IgA Nephropathy | 21 | 9 |
| Membranous | 0 | 7 |
| Nephropathy | | |
| Interstitial Nephritis | 6 | 8 |
| Minimal Change | 5 | 1 |
| Disease | | |
| Primary FSGS | 0 | 4 |
| Diabetic Nephropathy | 5 | 3 |
| Vasculitis | 5 | 3 |
| Hypertensive/ | 2 | 5 |
| ischaemic nephropathy | | |
| Lupus nephritis | 2 | 3 |
| Other * | 5 | 10 |

* Other diagnoses in discovery cohort: Henoch-Schönlein purpura (n = 2), lithium toxicity, obesity glomerulopathy and myeloma, and validation cohort: mesangiocapilllary glomerulonephritis (n = 2), thin glomerular basement membrance (n = 2), Henoch-Schönlein purpura, AA amyloid, AL amyloid, inflitration by lymphoma, CKD of uncertain aietiology and acute kidney injury.

Histological tubular senescence was quantified using a triple immunofluorescent stain (FIG. 1A). Tubular cells were classified as growth arrested if they were positive for p21CIP1 and negative for Ki67 and expressed as a percentage of all tubular cells. Median tubular senescence percentage was 5.2% in the discovery cohort (IQR 3.6-9.7%, range 1.6-20.1%) and 5.2% in the validation cohort (IQR 2.5-8.1%, range 0.8-21.2%). There was no statistical difference in senescence levels between the discovery and validation cohorts (p=0.3).

There was a strong correlation between tubular senescence percentage and age (rho=0.61 p<0.001) and inverse correlation with baseline eGFR (rho=−0.51 p<0.001). There was a weaker but significant relationship between tubular senescence and ACR (rho=0.24, p=0.015, FIG. 1B).

In the discovery cohort of n=51, LC-MS studies were conducted on paired urine samples. 331 proteins were detected. Levels were corrected for urinary creatinine concentration and correlated with tubular senescence percentage. Following adjustment for multiple testing, 143 proteins had a significant positive correlation with histological tubular senescence (adjusted p<0.05). Of these, 8 had a correlation coefficient (rho)>0.5 (Table 2). The SASP atlas of proteins secreted by senescent renal epithelia in vitro was used for additional validation. Of the 8 molecules with the strongest correlation, only Clusterin was upregulated in senescent renal epithelial cells in the SASP atlas.

TABLE 2
Spearman rank correlation

| | | | Adjusted |
|---|---|---|---|
| Protein | Rho | p value | p value |
| COMP | 0.580 | 0.000008 | 0.001 |
| PLG | 0.559 | 0.000020 | 0.001 |
| APOH | 0.534 | 0.000053 | 0.001 |
| C8A | 0.546 | 0.000035 | 0.001 |
| CLU | 0.534 | 0.000055 | 0.001 |
| HPX | 0.514 | 0.000116 | 0.002 |
| C7 | 0.515 | 0.000112 | 0.002 |
| TTR | 0.500 | 0.000190 | 0.002 |

Clusterin was tested for again in the discovery cohort samples using an ELISA and continued to correlate significantly with histological senescence (rho=0.54, p<0.001, FIG. 1C). Importantly, in a linear regression model, log transformed Clusterin:Creatinine predicted tubular senescence percentage, after adjusting for eGFR, ACR and age (Table 3).

Clusterin was then quantified in the validation cohort (n=53) using ELISA. Again, urinary Clusterin:Creatinine correlated strongly with tubular senescence (rho=0.61, p<0.001, FIG. 1D). Furthermore, Clusterin to creatinine ratio levels continued to predict tubular senescence after adjusting for eGFR, ACR and age in the validation cohort (Table 3).

TABLE 3
Linear regression models for urinary Clusterin:Creatinine
predicting levels of senescence after adjusting
for other patient characteristics.

| | Estimate | p value |
|---|---|---|
| Discovery cohort | | |
| Log₂ (Clusterin:creatinine ratio) | 0.597 | 0.039 |
| Baseline eGFR (mls/min) | −0.026 | 0.117 |
| Age (years) | 0.01 | 0.004 |
| ACR (mg/mmol) | 0.0001 | 0.798 |
| Validation cohort | | |
| Log₂ (Clusterin:creatinine ratio) | 1.47 | 0.005 |
| Baseline eGFR (mls/min) | −0.04 | 0.05 |
| Age (years) | 0.07 | 0.08 |
| ACR (mg/mmol) | 0.0001 | 0.96 |

Next, we generated a urinary Clusterin threshold, that could be used to find patients with a high burden of renal tubular senescence (defined as the highest tertile for histological tubular senescence percentage, FIG. 1E). We selected two possible values; 1. 124.5 μg/mmol, which had a sensitivity of 68% and specificity of 90% and 2. 58 μg/mmol, which had a sensitivity of 82% and specificity of 61%.

We then determined if urinary levels of Clusterin predicted decline in renal function in patients with CKD. We tested 322 urine samples in the seNSOR biobank from participants at moderate to high risk of renal progression (baseline eGFR<60 mls/min/1.73 m2 and/or ACR>30 mg/mmol, FIG. 1F, Table 4). Participants were followed up for 3 years following recruitment. The composite CKD progression endpoint (defined as reaching ESKD or >40% reduction in renal function from eGFR at baseline) occurred in 47 participants during follow-up.

TABLE 4
Baseline characteristics for cohort of
patients included in outcome analysis

| | Outcome cohort | |
|---|---|---|
| N | 322 | |
| Age (years), median (IQR). | 60.5 | (47.9, 69.3) |
| Male/Female, n | 192/130 | |
| eGFR (ml/min/1.73 m²), median (IQR) | 42.1 | (29.8, 64.4) |
| SBP, median (IQR) | 135 | (122, 148) |
| DBP, median (IQR) | 78 | (70, 84) |
| uACR (mg/mmol), median (IQR) | 81.6 | (14.5, 288.8) |

Ethnicity

| | |
|---|---|
| White, n | 279 |
| Black, n | 5 |
| Asian, n | 12 |
| Not recorded, n | 26 |

Diagnoses

| Glomerular Disease | 153 |
|---|---|
| Tubulointerstitial Disease | 44 |
| Diabetes Mellitus | 29 |
| Renovascular disease/Hypertension | 27 |
| Other systemic diseases affecting | 7 |
| kidney | |

| | |
|---|---|
| Familial/Hereditary Nephropathies | 10 |
| Miscellaneous Renal Disorders | 52 |

76 participants had

Clusterin:Creatinine results greater 124.5 μg/mmol. These participants had a significantly higher risk of CKD progression (log rank p<0.001; FIG. 1G). In Cox Proportional Hazards analysis, this remained significant, after adjusting for baseline eGFR, ACR, age, SBP and sex in multivariate analysis (HR 2.2, 95% C.I. 1.07-4.66, p=0.03, Table 5).

154 participants had Clusterin:Creatinine results greater 58 μg/mmol. These participants had a significantly higher risk of CKD progression (log rank p<0.001; FIG. 1H). In Cox Proportional Hazards analysis, this remained significant, after adjusting for baseline eGFR, ACR, age, SBP and sex in multivariate analysis (HR 2.2, 95% C.I. 1.02-4.69, p=0.04, Table 5).

Including Clusterin:Creatinine as continuous variables (either untransformed or log transformed) in models was also significant for predicting CKD progression in multivariate analyses.

TABLE 5
Results of Cox proportional hazards regression

| | | | |
|---|---|---|---|
| Hazard | | | |
| ratio | p | Lower CI | Upper CI |

Clusterin - using 124.5
μg/mmol threshold

| | | | | |
|---|---|---|---|---|
| Clusterin threshold (above | 2.234 | 0.032 | 1.071 | 4.662 |
| vs below 124.5 μg/mmol) | | | | |
| Baseline eGFR (mls/min) | 0.979 | 0.004 | 0.966 | 0.994 |
| LN (Albumin Creatinine | 1.254 | 0.043 | 1.007 | 1.562 |
| Ratio) | | | | |
| Age (years) | 0.976 | 0.034 | 0.955 | 0.998 |
| Systolic Blood Pressure | 1.018 | 0.012 | 1.004 | 1.032 |
| Sex (F vs M) | 1.022 | 0.945 | 0.552 | 1.893 |

Clusterin - using 58
μg/mmol threshold

| | | | | |
|---|---|---|---|---|
| Clusterin threshold (above | 2.188 | 0.044 | 1.021 | 4.686 |
| vs below 58 μg/mmol) | | | | |
| Baseline eGFR (mls/min) | 0.977 | 0.002 | 0.964 | 0.991 |
| LN (Albumin Creatinine | 1.272 | 0.028 | 1.026 | 1.576 |
| Ratio) | | | | |
| Age (years) | 0.976 | 0.028 | 0.955 | 0.997 |
| Systolic Blood Pressure | 1.019 | 0.007 | 1.005 | 1.033 |
| Sex (F vs M) | 0.956 | 0.887 | 0.513 | 1.780 |

Clusterin - using
continuous values

| | | | | |
|---|---|---|---|---|
| Clusterin:Creatinine ratio | 1.060 | 0.002 | 1.022 | 1.099 |
| per 100 μg/mmol | | | | |
| Baseline eGFR (mls/min) | 0.977 | 0.002 | 0.964 | 0.991 |
| LN (Albumin Creatinine | 1.321 | 0.005 | 1.090 | 1.602 |
| Ratio) | | | | |
| Age (years) | 0.974 | 0.021 | 0.952 | 0.996 |
| Systolic Blood Pressure | 1.020 | 0.005 | 1.006 | 1.033 |
| Sex (F vs M) | 1.102 | 0.759 | 0.593 | 2.049 |

Clusterin - using
log tranformed values

| | | | | |
|---|---|---|---|---|
| Log2 (Clusterin:Creatinine) | 1.33 | 0.01 | 1.07 | 1.64 |
| Baseline eGFR (mls/min) | 0.98 | 0.01 | 0.97 | 0.99 |
| LN (Albumin Creatinine | 1.15 | 0.24 | 0.91 | 1.46 |
| Ratio) | | | | |
| Age (years) | 0.98 | 0.02 | 0.95 | 1.00 |
| Systolic Blood Pressure | 1.02 | 0.01 | 1.00 | 1.03 |
| Sex (F vs M) | 1.07 | 0.84 | 0.57 | 1.99 |

Here, we utilised proteomic analysis of human urine samples to identify Clusterin as a biomarker of renal senescence. This was validated in a separate cohort and elevated urinary Clusterin levels had a high specificity for finding those with a high burden of renal senescence. This could be used as a tool to enrich clinical trials of senolytic therapies with participants both most in need of treatment, due to the increased risk of CKD progression, but also most likely to benefit from it.

To seek further evidence of CLU (codes for Clusterin protein) expression in senescent renal epithelial in vitro, we performed cultures using primary human renal proximal tubular epithelial cells (hRPTECs). Irradiation-induced senescence is an established model for studying senescence and here the senescent cells demonstrated decreased proliferation, with reduced MKI67 (p<0.0005 vs proliferating controls) and increased CDKN1A (confirming they are senescence) but also increase CLU (p<0.05 vs proliferating controls) (FIG. 6).

Furthermore, to determine if Clusterin production was selectively increased in senescent cells at a transcript level in human kidney biopsies, we performed ×1000 plex spatial transcriptomic analysis using the CosMx platform on kidney tissue from 13 patients with CKD. CLU transcript levels were enriched within CDKN1A-expressing proximal tubular epithelia (CKDN1A codes for p21, which is a marker of senescence) on automated counting and co-localisation analysis (n=114,135 epithelia, pAdj=3.91×10-8, FIG. 7). FIG. 7 shows CLU/CDKN1A co-localisation by spatial transcriptomic analysis and Spatial enrichment (log 2 scale) of CLU transcripts in proximity to P21+ and P21-renal proximal tubule (PT) or loop of Henle/distal convoluted tubule (LOH/DCT) epithelial cells.

Importantly, it was confirmed that this pattern is also evident at a protein level. In immunofluorescent staining and analysis of n=5 obstructed human kidneys, Clusterin positivity was 66% higher in p21 positive cells compared to p21 negative cells (n=230,028 epithelia, p<0.0001 by Fisher's test) (FIG. 8).

### EXAMPLE 2—URINARY FIBRONECTIN 1 PREDICTS LEVELS OF RENAL SENESCENCE

Using a similar approach exemplified for Clusterin, Fibronectin 1 was tested for in the discovery cohort samples using an ELISA and correlated significantly with histological senescence (rho=0.64, p<0.001) (FIG. 2).

Importantly, in a linear regression model, log transformed Fibronectin 1:Creatinine predicted tubular senescence percentage, after adjusting for eGFR, ACR and age (Table 6).

TABLE 6
Linear regression models for urinary Fibronectin 1: Creatinine
predicting levels of senescence after adjusting for other
patient characteristics - Discovery cohort.

| Discovery cohort | Estimate | p value |
|---|---|---|
| Log₂ (Fibronectin 1 to creatinine ratio) | 1.09 | 0.008 |
| Baseline eGFR (mls/min) | −0.02 | 0.15 |
| Age (years) | 0.08 | 0.02 |
| ACR (mg/mmol) | 0.002 | 0.35 |

Fibronectin 1 was then quantified in the validation cohort (n=53) using ELISA. Again, urinary Fibronectin 1:Creatinine correlated strongly with tubular senescence (rho=0.62, p<0.001) (FIG. 3).

Furthermore, Fibronectin 1 to creatinine ratio levels continued to predict tubular senescence after adjusting for eGFR, ACR and age in the validation cohort (Table 7).

TABLE 7
Linear regression models for urinary Fibronectin 1: Creatinine
predicting levels of senescence after adjusting for other
patient characteristics - Validation cohort.

| Validation cohort | Estimate | p value |
|---|---|---|
| Log₂ (Fibronectin 1 to creatinine ratio) | 1.94 | 0.001 |
| Baseline eGFR (mls/min) | −0.02 | 0.21 |
| Age (years) | 0.06 | 0.106 |
| ACR (mg/mmol) | 0.002 | 0.39 |

Next, we generated a urinary Fibronectin 1 threshold, that could be used to find patients with a high burden of renal tubular senescence (defined as the highest quartile for histological tubular senescence percentage). We selected 32.1 μg/mmol, which had a sensitivity of 77% and specificity of 95% and 2. 40 μg/mmol, which had a sensitivity of 62% and specificity of 95% (FIG. 4).

We then determined if urinary levels of Fibronectin 1 predicted decline in renal function in patients with CKD. We tested 322 urine samples in the seNSOR biobank from participants at moderate to high risk of renal progression (baseline eGFR<60 mls/min/1.73 m2 and/or ACR>30). Participants were followed up for 3 years following recruitment. The composite CKD progression endpoint (defined as reaching ESKD or >40% reduction in renal function from eGFR at baseline) occurred in 47 participants during follow-up. 69 participants had Fibronectin 1:Creatinine results greater 32.1 μg/mmol. These participants had a significantly higher risk of CKD progression (log rank p<0.001) (FIG. 5).

56 participants had Fibronectin 1:Creatinine results greater 40 μg/mmol. These participants had a significantly higher risk of CKD progression (log rank p<0.001) (FIG. 9). In Cox Proportional Hazards analysis (Table 8), this remained significant after adjusting for baseline eGFR, ACR, age, SBP and sex in multivariate analysis (HR 2.2, 95% C.I. 1.05-4.53, p=0.04). Including Fibroectin:Creatinine as continuous variables (either untransformed or log transformed) in models was also significant for predicting CKD progression in multivariate analyses.

TABLE 8
Results of Cox proportional hazards regression

| | | | |
|---|---|---|---|
| Hazard | | Lower | Upper |
| ratio | p | CI | CI |

Fibronectin - using 32.1
μg/mmol threshold

| | | | | |
|---|---|---|---|---|
| Fibronectin 1 threshold (above vs below | 1.660 | 0.154 | 0.827 | 3.330 |
| 32.1 μg/mmol) | | | | |
| Baseline eGFR (mls/min) | 0.979 | 0.004 | 0.965 | 0.993 |
| LN (Albumin Creatinine Ratio) | 1.351 | 0.004 | 1.099 | 1.659 |
| Age (years) | 0.976 | 0.030 | 0.955 | 0.998 |
| Systolic Blood Pressure | 1.019 | 0.007 | 1.005 | 1.033 |
| Sex (F vs M) | 1.084 | 0.797 | 0.585 | 2.009 |

Fibronectin - using 40
μg/mmol threshold

| | | | | |
|---|---|---|---|---|
| Fibronectin 1 threshold (above vs below | 2.183 | 0.036 | 1.051 | 4.534 |
| 40 μg/mmol) | | | | |
| Baseline eGFR (mls/min) | 0.980 | 0.008 | 0.966 | 0.995 |
| LN (Albumin Creatinine Ratio) | 1.297 | 0.014 | 1.055 | 1.595 |
| Age (years) | 0.976 | 0.033 | 0.955 | 0.998 |
| Systolic Blood Pressure | 1.018 | 0.009 | 1.004 | 1.032 |
| Sex (F vs M) | 1.149 | 0.663 | 0.616 | 2.143 |

Fibronectin 1 - using continuous values

| | | | | |
|---|---|---|---|---|
| Fibronecin 1:Creatinine ratio per | 1.057 | 0.0001 | 1.028 | 1.088 |
| 10 μg/mmol | | | | |
| Baseline eGFR (mls/min) | 0.980 | 0.007 | 0.966 | 0.994 |
| LN (Albumin Creatinine Ratio) | 1.328 | 0.001 | 1.119 | 1.576 |
| Age (years) | 0.974 | 0.022 | 0.952 | 0.996 |
| Systolic Blood Pressure | 1.019 | 0.005 | 1.006 | 1.032 |
| Sex (F vs M) | 1.109 | 0.744 | 0.596 | 2.063 |

Fibronectin 1 - using log transformed values

| | | | | |
|---|---|---|---|---|
| Log2 (Fibronectin 1:Creatinine) | 1.46723 | 0.00092 | 1.16955 | 1.84067 |
| Baseline eGFR (mls/min) | 0.98217 | 0.0167 | 0.96781 | 0.99675 |
| LN (Albumin Creatinine Ratio) | 1.23488 | 0.02999 | 1.02065 | 1.49408 |
| Age (years) | 0.97151 | 0.01178 | 0.9499 | 0.99361 |
| Systolic Blood Pressure | 1.01774 | 0.00823 | 1.00455 | 1.03111 |
| Sex (F vs M) | 1.15187 | 0.65932 | 0.61435 | 2.15968 |

### Materials and Methods

seNSOR Biobank Recruitment

The seNSOR biobank comprises 635 patients recruited from renal clinics at the Royal Infirmary of Edinburgh, Edinburgh between March 2017 and March 2019. In addition, 100 patients with CKD were recruited to seNSOR from the Queen Elizabeth University Hospital, Glasgow between March 2018 and August 2019. Ethical approval was obtained from the Offices for Research Ethics Committee (REC/15/ES/0094, REC/20/ES/0061, REC 14/WS/1035 and REC 22/WS/0020) and informed patient consent and anonymisation undertaken in line with the Uniform Requirements of the International Committee of Medical Journal Editors. Participant information was collected upon enrolment with urine samples snap frozen and stored at −80° C.

### Patient Selection for Discovery and Validation Cohorts

The discovery cohort of 51 participants included all those recruited in Edinburgh that matched kidney tissue and urine samples available for analysis. The validation cohort included 53 participants that were recruited in Glasgow and also had matched kidney tissue and urine samples available.

### Patient Selection for Outcome Analysis

Urine samples were available from 570 participants recruited into the seNSOR. 129 patients at low risk of CKD progression (baseline eGFR>60 mls/min and ACR<30 mg/mmol) were excluded. This matches the criterion used by other CKD biobanks, including The National Unified Renal Translational Research Enterprise (NURTURE) biobank, which is the largest in the UK. An additional 119 with, or close to, ESKD (baseline eGFR<20 mls/min) where excluded. The remaining 322 participants were included in the outcome analysis.

### LC-MS Analysis

LC-MS studies were undertaken on all urine samples in the discovery cohort by Lisa Imrie and Tessa Moses from the Edinomics Team (University of Edinburgh). Samples with depleted of high abundance proteins (serum albumin and IgG) using Agilent multiple affinity removal spin (MARS) cartridges following manufacturers protocol. They were trypsin digested using S-Trap™ (Protifi) following manufacturers protocol. After speed vac drying, peptide samples were re-suspended in MS-loading buffer (0.05% v/v trifluoroacetic acid in water) and 50 pmol of MassPREP Alcohol dehydrogenase (ADH) digestion standard (Waters) was spiked into each sample (added as an external standard). They were then filtered using Millex filter before HPLC-MS analysis.

Nano-Electrospray ionization (ESI)-High-performance liquid chromatography (HPLC)-MS/MS analysis was performed using an online system of a nano-HPLC (Dionex Ultimate 3000 RSLC, Thermo-Fisher Scientific) coupled to a QExactive mass spectrometer (Thermo-Fisher Scientific) with a 300 μm×5 mm pre-column (Acclaim Pepmap, 5 μm particle size) joined with a 75 μm×50 cm column (EASY-Spray, 3 μm particle size). The nano-pump was run using solvent A (2% Acetonitrile in water 0.1% formic acid) and solvent B (80% acetonitrile-20% water and 0.1% formic acid) and peptides were separated using a multi-step gradient of 2-98% buffer B at a flow rate of 300 nL/min over 90 min. Progenesis (version 4 Nonlinear Dynamics, UK) was used for LC-MS label-free quantitation. Filtering was carried out so that only MS/MS peaks with a charge of 2+, 3+ or 4+ were taken into account for the total number of ‘features’ (signal at one particular retention time and m/z) and only the five most intense spectra per ‘feature’ were included. MS/MS spectra was searched using MASCOT Version 2.4 (Matrix Science Ltd) against a UniProt H. sapiens database with maximum missed-cut value set to 2. The following parameters were used in all searches: i) variable methionine oxidation, ii) fixed cysteine carbamidomethylation, iii) precursor mass tolerance of 10 ppm, iv) MS/MS tolerance of 0.05 Da, v) significance threshold (p) below 0.05 and vi) final peptide score of 20. Only proteins with 2 or more unique peptides were considered.

### ELISA Analysis

Urine Clusterin was measured on stored urine samples using R&D Duoset ELISAs (R&D Systems, Minneapolis, MN, catalogue number DY5874) with all samples run in duplicate. Based on pilot studies most samples were analysed following 1:1000 pre-dilution, with any results outside the published working range of the assay rerun at 1:100, 1 in 10,000 or 1:100,000 dilution.

Fibronectin 1 used R&D duoset ELISA catalogue number DY1918-05. Based on pilot studies most samples were analysed following 1 in 10 dilution. Any results outside the published working range of the assay rerun neat or at a 1:2, 1:3 or 1:100 dilution

### Biochemical Assays

Urinary creatinine measurements were determined using the creatininase/creatinase enzymatic method making use of a commercial kit (17654H, Sentinel Diagnostics via Alpha Laboratories Ltd., Eastleigh, UK) adapted for use on either a Cobas Fara or Mira analyser (Roche). Intra-assay precision was <3% while inter-assay precision was CV<5%. Urinary Clusterin and Fibronectin 1 levels were corrected for urinary creatinine throughout.

Microalbumin measurements were determined using a commercial kit (#1 0242 99 10 021, DiaSys Diagnostic Systems) adapted for use on a Cobas Mira analyser (Roche). This immunoturbidimetric assay was standardised against purified mouse albumin standards (Sigma Aldrich) with samples diluted in deionised water as appropriate. Intra-assay precision was <5% while inter-assay precision was <7.1%.

### Tissue Biopsy Staining

Immunofluorescence staining on human kidney biopsy samples was performed using a BOND III automated immunostainer (Leica Biosystems) using sequential Tyramide-coupled fluorophores. Antibody concentrations were determined by titration of single immunofluorescent stains to determine the most suitable dilution before being combined into a multiplex stain. The immunofluorescent stain comprised 4 antibodies. Pancytokeratin (CKPAN, Merck, Cat C2562) at 1:6000 dilution, and CD10 (Leica, Cat: NCL-L-CD10-270) at 1:600, both tubular epithelial markers, were added together, and the same Opal 520 fluorophore used for both. Other antibodies used in sequence targeted Ki67 (Agilent, Cat M724001-2) with an Opal 650 fluorophore and p21 (Abcam, Cat ab109520) with an Opal 570 fluorophore (all secondary antibodies used at 1:500 dilution). For the P21/Clusterin staining, Clusterin (Abcam, Cat ab92548) was substituted into the panel at 1:500 dilution in place of KI67 (using the Opal 650 fluorophore) with P21, Pancytokeratin and CD10 included as described above.

### Microscopy and Image Analysis

Images of whole slides were acquired using the Axio Scan Z1 whole slide scanner (Zeiss, Jena, Germany). Images were then analysed using QUPath (version 0.3.2). Cells were classified automatically using the algorithm below based on nuclear staining of p21 and Ki67 as follows; p21 positive/Ki67 negative, Ki67 positive/p21negative, double positive or double negative. Only tubular epithelia that were p21 positive and Ki67 negative were classed as having a cell cycle inhibitor profile consistent with senescence and this number was expressed as a percentage of all tubular cells. In participants where >1 section from their biopsy was available, the cell counts from each section were combined before percentages were calculated.

For the P21/Clusterin staining, images were acquired using the Zeiss Observer scanner (Zeiss, Jena, Germany). Images were then analysed using QUPath (version 0.5.1). Cells were classified based on nuclear staining of P21 and cytoplasmic staining for Clusterin.

hRPTEC Cell Culture

Human renal proximal tubular epithelial cells (hRPTEC) (American Type Culture Collection (ATCC)) were maintained in Dulbecco's modified Eagle's medium/F-12 with GlutaMAX (Thermo Fisher, Cat 31331028) and supplemented with a human telomerase reverse transcriptase (hTERT) immortalized RPTEC growth kit (ATCC, ACS-4007), Pen-Strep and Geneticin (50 mg/ml) (Thermo Fisher, Cat 10131035). Cells were grown at 37° C. in 5% CO₂. hRPTECs were plated in twelve-well culture plates at 2.4×10⁴ cells (control group) or 4.8×10⁴ cells (irradiated group) per well. After 72 hours of culture, hRPTECs were exposed to 10-Gy radiation. Control hRPTECs were taken to the irradiator but not placed inside. Culture medium was changed immediately following irradiation then every 2-3 days. 7 days after irradiation, RNA was extracted with TRIzol.

hRPTEC RNA Extraction and qPCR

RNA was extracted from hRPTECs using TRIzol (Thermo Fisher, Cat 15596026) and reverse transcribed to cDNA with QuantiTect Reverse Transcription Kit (Qiagen, Cat 205314) according to manufacturer's instructions. qPCR was carried out using PerfeCTa FastMix II (Quantabio, Cat 95118-012) and TaqMan Gene Expression Assays (Thermo Fisher, Cat 4331182) on a QuantStudio™ 5 Real-Time PCR instrument. TaqMan assays used were: CDKN1A (Hs00355782_m1), CLU (Hs00156548_m1), HPRT1 (Hs02800695_m1), MKI67 (Hs04260396_g1) and PPIA (Hs04194521_s1). mRNA expression was normalised for HPRT1 and PPIA (average) expression and presented as relative expression (2^{ΔΔCt}) to nonirradiated, proliferating controls.

### Spatial Transcriptomic Analysis

Spatial transcriptomic analysis was performed on kidney tissue from 13 patients; this included 9 core biopsies from patients in the discovery cohort (n=3 with minimal change disease and n=6 with IgA nephropathy) and an additional 4 nephrectomy specimens from patients with fibrosis due to recurrent pyelonephritis (Table S1B). CDKN1A and CLU expression in sub-cellular resolution spatial transcriptomics data (Bruker Spatial Biology CosMx SMI) was analysed using pre-processed data downloaded in Seurat (4.4.0) format from gene expression omnibus (GSE253439). The original cell annotations were used to classify proximal tubule (PT), or loop of Henle and distal convoluted tubule (LOH-DCT) cells which express CDKN1A (independent of cell state).

To avoid false positive classifications due to noise in the assay and cell segmentation errors, cells were considered CDKN1A+ when at least 2 or more CDKN1A transcripts were detected within cell segmentation boundaries.

Spatial enrichment of CLU transcripts in relation to cell centroids of a given cell type were calculated as described previously (5). Briefly, for each individual cell, a search radius of 50 μm in 1 μm steps from the cell centroid was defined. For each given cell type, the number of detected transcripts (of a given gene) were summed and normalised by the area of the circle segment and the number of cells encountered in the search area. Simultaneously, to calculate the background signal in the general cell population, 10,000 random cells were selected and the normalised transcript counts were calculated as before. The enrichment ratio at each interval was then defined as the log 2+1-fold enrichment ratio of the query cell type over the randomly selected cell population. Cell boundaries and transcripts in 2D coordinates were visualised using the Seurat ImageDimPlot function.

Differential gene expression between CDKN1A+ and CDKN1A-PT cells was assessed using the Wilcoxon signed-rank test implemented by the Seurat function FindMarkers( ) with default parameters.

### Statistical Tests

Normality was assessed by Shapiro-Wilk test for all variables. Clinical characteristics for continuous data were expressed as mean±standard deviation when data was normally distributed and median (interquartile range) when not normally distributed. Categorical variables were expressed as counts. When comparing two unpaired groups, a T-test was used when the data was normally distributed, and a Mann-Whitney test used if the data was not normally distributed. Categorical values were assessed using a Chi-square test.

For the LC-MS data, values were corrected for ADH and then for urinary creatinine. To determine the linear correlation between each protein detected and histological p21+Ki67− epithelial cell proportions, correlation coefficients (rho) were estimated using Spearman's rank tests as the data was not normally distributed. Adjusted p values were calculated using the False Discovery Rate (FDR) approach from the list of p values, generated from the correlation between p21+Ki67− epithelial cell proportions and protein levels.

For qPCR analysis of in vitro cell culture data, relative expression of CDKN1A, MKI67 and CLU in irradiated hRPTECs was compared to proliferating controls, normalised for HPRT1 and PPIA (average) expression and Welch's t-test performed.

Linear regression was used to determine if levels of log 2-transformed Clusterin and Fibronectin 1 predicted histological p21+Ki67− epithelial cell proportions as the dependent variable in models alongside baseline eGFR, ACR and patient age.

Receiver operating characteristic (ROC) curve analysis was used to explore discrimination between those with a level of senescence in the top tertile and determine the optimal cutoff point for the top performing biomarkers.

For the outcome analysis, CKD progression was defined as reaching ESKD (starting renal replacement therapy (RRT) or maintaining an eGFR<15 mls/min for >90 days) or >40% reduction in renal function from eGFR at baseline (maintained for >90 days), which was based on the 2020 ISN consensus definition of an adverse renal outcome. Kaplan-Meier survival curves were constructed with the log-rank test used to compare curves. Univariate and multivariate analyses of outcomes using Cox proportional hazards survival models were performed. Death was treated as censoring event. The proportional hazards assumption was tested and valid. A p value of less than 0.05 was considered significant.

The present disclosure will now be further described, by way of example only, with reference to the following clauses:
Clause 1. Use of Clusterin and/or Fibronectin 1 as renal cellular senescence biomarker(s) for evaluating chronic kidney disease (CKD), wherein the level of Clusterin and/or Fibronectin 1 in a fluid sample from a subject is indicative of renal senescent cell load of said subject.
Clause 2. The use according to clause 1, wherein the fluid sample is a urine sample and/or a blood sample.
Clause 3. The use according to any of clauses 1 to 2, wherein the sample is a urine sample.
Clause 4. The use according to any preceding clause, wherein the subject is a mammalian subject.
Clause 5. The use according to any preceding clause, wherein the level of Clusterin and/or Fibronectin 1 in said fluid sample indicative of:
   (i) decline in renal function;
   (ii) the likelihood of developing chronic kidney disease;
   (iii) the severity of chronic kidney disease;
   (iv) progression of chronic kidney disease;
   (v) the likelihood of progression of chronic kidney disease; and/or
   (vi) the efficacy or effectiveness of a treatment, therapy or drug for chronic kidney disease.
Clause 6. The use according to any preceding clause, wherein the renal senescent cell load is renal tubular epithelial senescent cell load.
Clause 7. The use according to any preceding clause further comprising up to 10 additional biomarkers.
Clause 8. The use according to any preceding clause further comprising up to 5 additional biomarkers.
Clause 9. The use according to any preceding clause further comprising 1, 2, 3, 4 or 5 additional biomarkers.
Clause 10. The use according to clauses 1 to 6, wherein said use consists of or consists essentially of Clusterin and/or Fibronectin 1.
Clause 11. A method of evaluating chronic kidney disease, said method comprising determining the level of one or more renal cellular senescence biomarkers in a fluid sample, wherein said one or more renal cellular senescence biomarkers comprise Clusterin and/or Fibronectin 1, and wherein the level of said one or more renal cellular senescence biomarkers is indicative of renal senescent cell load.
Clause 12. The method according to clause 11, wherein the fluid sample is a urine sample and/or a blood sample.
Clause 13. The method according to any of clauses 11 to 12, wherein the sample is a urine sample.
Clause 14. The method according to any of clauses 11 to 13, wherein the level one or more biomarkers is evaluated with reference to a threshold.
Clause 15. The method according to clause 14, wherein the threshold comprises, consists essentially of or consists of:
   120-160 μg/mmol for Clusterin (μg Clusterin per mmol Creatinine); and/or
   30-50 μg/mmol for Fibronectin 1 (μg Fibronectin 1 per mmol Creatinine).
Clause 16. The method according to any of clauses 11 to 15, wherein the level of one or more renal cellular senescence biomarkers is indicative of:
   (i) decline in renal function;
   (ii) the likelihood of developing chronic kidney disease;
   (iii) the severity of chronic kidney disease;
   (iv) progression of chronic kidney disease;
   (v) the likelihood of progression of chronic kidney disease; and/or
   (vi) the efficacy or effectiveness of a treatment, therapy or drug for a kidney disease.
Clause 17. The method according to any of clauses 11 to 16 further comprising determining the level of up to 10 additional biomarkers.
Clause 18. The method according to any of clauses 11 to 17 further comprising determining the level of up to 5 additional biomarkers.
Clause 19. The method according to any of clauses 11 to 18 further comprising determining the level of 1, 2, 3, 4 or 5 additional biomarkers.
Clause 20. The method according to any of clauses 11 to 16, wherein the one or more renal cellular senescence biomarkers consist of or consist essentially of Clusterin and/or Fibronectin 1.
Clause 21. A kit for evaluating chronic kidney disease, said kit comprising one or more probes for the detection of one or more renal cellular senescence biomarkers, wherein said one or more renal cellular senescence biomarkers comprise Clusterin and/or Fibronectin 1.
Clause 22. The kit according to clause 21 further comprising one or more positive and/or negative controls.
Clause 23. The kit according to any of clauses 21 to 22 further comprising a receptacle for a urine sample and/or a blood sample.
Clause 24. The kit according to any of clauses 21 to 23 further comprising a receptacle for a urine sample.
Clause 25. The kit according to any of clauses 21 to 24, wherein the kit comprises one or more probes for the detection of up to 10 additional biomarkers.
Clause 26. The kit according to any of clauses 21 to 25, wherein the kit comprises one or more probes for the detection of up to 5 additional biomarkers.
Clause 27. The kit according to any of clauses 21 to 26, wherein the kit comprises one or more probes for the detection of 1, 2, 3, 4 or 5 additional biomarkers.
Clause 28. The kit according to any of clauses 25 to 27, wherein the additional biomarkers are selected from:
   (i) Cystatin C;
   (ii) Kidney injury molecule-1 (KIM1);
   (iii) Epidermal growth factor (EGF);
   (iv) Monocyte chemoattractant protein 1;
   (v) Transforming growth factor alpha;
   (vi) Neutrophil Gelatinase-associated Lipocalin (NGAL);
   (vii) Growth and Differentiation Factor 15 (GDF15);
   (viii) Fatty Acid Binding Protein (FABP);
   (ix) Osteopontin;
   (x) Tissue Inhibitor of Mettaloprotease 1 (TIMP1);
   (xi) Uromodulin;
   (xii) Vascular Endothelial Growth Factor A (VEGFA);
   (xiii) Interleukin-6 (IL6);
   (xiv) Leukemia Inhibitory Factor (LIF);
   (xv) Matrix Metallopeptidase 9 (MMP9);
   (xvi) Albumin;
   (xvii) Creatinine.
Clause 29. The kit according to any of clauses 21 to 24, wherein said one or more renal cellular senescence biomarkers consist of or consist essentially of Clusterin and/or Fibronectin 1.
Clause 30. The kit according to any of clauses 21 to 29, wherein said kit comprises, consists essentially of or consists of any one or more of the following components:
   (i) a lateral flow device or a plate for an immunoassay, wherein said device or plate comprises one or more antibodies for the detection of Clusterin and/or Fibronectin 1; optionally, wherein said device or plate further comprises one or more antibodies for the detection of one or more additional biomarkers;
   (ii) a negative control;
   (iii) a positive control;
   (iv) a receptacle;
   (v) one or more solution(s) and/or buffer(s).
Clause 31. The kit according to any of clauses 21 to 30, wherein the detection of one or more renal cellular senescence biomarkers is indicative of:
   (i) decline in renal function;
   (ii) the likelihood of developing chronic kidney disease;
   (iii) the severity of chronic kidney disease;
   (iv) progression of chronic kidney disease;
   (v) the likelihood of progression of chronic kidney disease; and/or
   (vi) the efficacy or effectiveness of a treatment, therapy or drug for a kidney disease.

### REFERENCES

1. Jager K J, Kovesdy C, Langham R, Rosenberg M, Jha V, Zoccali C. A single number for advocacy and communication-worldwide more than 850 million individuals have kidney diseases. Kidney Int. 2019; 96(5):1048-50.
2. Docherty M H, Baird D P, Hughes J, Ferenbach D A. Cellular Senescence and Senotherapies in the Kidney: Current Evidence and Future Directions. Frontiers in Pharmacology. 2020; 11(755).
3. Mylonas K J, O'Sullivan E D, Humphries D, Baird D P, Docherty M H, Neely S A, et al. Cellular senescence inhibits renal regeneration after injury in mice, with senolytic treatment promoting repair. Sci Transl Med. 2021; 13(594).
4. Basisty N, Kale A, Jeon O H, Kuehnemann C, Payne T, Rao C, et al. A proteomic atlas of senescence-associated secretomes for aging biomarker development. PLOS Biol. 2020; 18(1):e3000599.

That which is claimed is:

1. A method of evaluating chronic kidney disease, said method comprising determining the level of one or more renal cellular senescence biomarkers in a fluid sample, wherein said one or more renal cellular senescence biomarkers comprise Clusterin and/or Fibronectin 1, and wherein the level of said one or more renal cellular senescence biomarkers is indicative of renal senescent cell load.

2. The method according to claim 1, wherein the fluid sample is a urine sample and/or a blood sample.

3. The method according to claim 1, wherein the sample is a urine sample.

4. The method according to claim 1, wherein the level one or more biomarkers is evaluated with reference to a threshold.

5. The method according to claim 4, wherein the threshold comprises, consists essentially of or consists of:
10-1000 μg/mmol for Clusterin (μg Clusterin per mmol Creatinine); and/or
15-260 μg/mmol for Fibronectin 1 (μg Fibronectin 1 per mmol Creatinine).

6. The method according to claim 1, wherein the level of one or more renal cellular senescence biomarkers is indicative of:
(i) decline in renal function;
(ii) the likelihood of developing chronic kidney disease;
(iii) the severity of chronic kidney disease;
(iv) progression of chronic kidney disease;
(v) the likelihood of progression of chronic kidney disease; and/or
(vi) the efficacy or effectiveness of a treatment, therapy or drug for a kidney disease.

7. The method according to claim 1 further comprising determining the level of up to 10 additional biomarkers.

8. The method according to claim 1 further comprising determining the level of up to 5 additional biomarkers.

9. The method according to claim 1 further comprising determining the level of 1, 2, 3, 4 or 5 additional biomarkers.

10. The method according to claim 1, wherein the one or more renal cellular senescence biomarkers consist of or consist essentially of Clusterin and/or Fibronectin 1.

11. A kit for evaluating chronic kidney disease, said kit comprising one or more probes for the detection of one or more renal cellular senescence biomarkers, wherein said one or more renal cellular senescence biomarkers comprise Clusterin and/or Fibronectin 1.

12. The kit according to claim 11 further comprising one or more positive and/or negative controls.

13. The kit according to claim 11 further comprising a receptacle for a urine sample and/or a blood sample.

14. The kit according to claim 11, wherein the kit comprises one or more probes for the detection of up to 10 additional biomarkers.

15. The kit according to claim 11, wherein the kit comprises one or more probes for the detection of up to 5 additional biomarkers.

16. The kit according to claim 11, wherein the kit comprises one or more probes for the detection of 1, 2, 3, 4 or 5 additional biomarkers.

17. The kit according to claim 14, wherein the additional biomarkers are selected from:
(i) Cystatin C;
(ii) Kidney injury molecule-1 (KIM1);
(iii) Epidermal growth factor (EGF);
(iv) Monocyte chemoattractant protein 1;
(v) Transforming growth factor alpha;
(vi) Neutrophil Gelatinase-associated Lipocalin (NGAL);
(vii) Growth and Differentiation Factor 15 (GDF15);
(viii) Fatty Acid Binding Protein (FABP);
(ix) Osteopontin;
(x) Tissue Inhibitor of Mettaloprotease 1 (TIMP1);
(xi) Uromodulin;
(xii) Vascular Endothelial Growth Factor A (VEGFA);
(xiii Interleukin-6 (IL6);
(xiv) Leukemia Inhibitory Factor (LIF);
(xv) Matrix Metallopeptidase 9 (MMP9);
(xvi) Albumin;
(xvii) Creatinine.

18. The kit according to claim 11, wherein said one or more renal cellular senescence biomarkers consist of or consist essentially of Clusterin and/or Fibronectin 1.

19. The kit according to claim 11, wherein said kit comprises, consists essentially of or consists of any one or more of the following components:
(i) a lateral flow device or a plate for an immunoassay, wherein said device or plate comprises one or more antibodies for the detection of Clusterin and/or Fibronectin 1; optionally, wherein said device or plate further comprises one or more antibodies for the detection of one or more additional biomarkers;
(ii) a negative control;
(iii) a positive control;
(iv) a receptacle;
(v) one or more solution(s) and/or buffer(s).

20. The kit according to claim 11, wherein the detection of one or more renal cellular senescence biomarkers is indicative of:
(i) decline in renal function;
(ii) the likelihood of developing chronic kidney disease;
(iii) the severity of chronic kidney disease;
(iv) progression of chronic kidney disease;
(v) the likelihood of progression of chronic kidney disease; and/or
(vi) the efficacy or effectiveness of a treatment, therapy or drug for a kidney disease.
